(19) United States
(12) Patent Application Publication
(10) Pub. No.: US 2025/0223635 A1
(43) Pub. Date: Jul. 10, 2025

(54) ULTRA-FAST ONE-POT LOOP-MEDIATED ISOTHERMAL AMPLIFICATION AND DETECTION OF DNA AND RNA

(71) Applicant: DOMUS DIAGNOSTICS, INC., Park City, UT (US)

(72) Inventors: Xin SONG, Raleigh, NC (US); John H. REIF, Durham, NC (US); Jacquelyn M. WALEJKO, Durham, NC (US)

(21) Appl. No.: 19/011,449

(22) Filed: Jan. 6, 2025

Related U.S. Application Data
(60) Provisional application No. 63/618,116, filed on Jan. 5, 2024.

(51) Int. Cl. C12Q 1/6844

(52) U.S. Cl. CPC C12Q 1/6844

(57) ABSTRACT

Disclosed are assay formulations and methods that enable ultra-fast (e.g., 5 min to 15 min turnaround time, depending on target concentration), one-pot detection of nucleic acids. The assay can implement an ultra-fast LAMP or RT-LAMP reaction with real-time fluorescent readout, pH-independent colorimetric readout, and/or pH-dependent colorimetric readout. The assay enables quantification of the target with fluorescent readout. The assay can be carried out in one-pot format with the reaction components in solution form, lyophilized form, or air-dried form. The assay enables a broader effective temperature range for the LAMP or RT-LAMP reaction as compared to conventional assay chemistries.

### CROSS-REFERENCE TO RELATED APPLICATIONS

This application claims priority to and the benefit of U.S. Provisional Patent Application No. 63/618,116, filed Jan. 5, 2024, the entirety of which is incorporated herein by reference.

### BACKGROUND

Availability of fast, precise, and cost-effective nucleic acid testing can help prevent future pandemics. Polymerase chain reaction (PCR and its variations such as qPCR, RT-PCR, RT-qPCR, etc.) has conventionally been used as the gold-standard method for nucleic acid detection. However, PCR-based molecular amplification methods rely on precise thermocycling reactions that require expensive instrumentation, trained personnel, and are often time-consuming.

Various isothermal amplification methods have been developed to eliminate the need for thermocycling. An example of such an isothermal amplification method is loop-mediated isothermal amplification (commonly referred to as LAMP for DNA detection, or RT-LAMP for RNA detection). This method employs a set of four to six primers and a strand-displacing DNA polymerase to amplify minute quantities of the target nucleic acid exponentially, maintaining a constant temperature throughout. Reaction time typically ranges from 30 minutes to one hour. For RNA detection, the reaction can use a strand-displacing DNA polymerase with sufficient reverse transcriptase activity or can additionally include a dedicated reverse transcriptase. Such molecular assays eliminate the need for expensive thermocycling instruments and exhibit better sensitivity and accuracy in contrast to rapid antigen tests for diagnosing infectious diseases.

Various readout methods have been suggested for LAMP and RT-LAMP, enabling their suitability for diagnostic application in at-home and point-of-care settings. See, for example: Zhang et al., Enhancing Colorimetric Loop-Mediated Isothermal Amplification Speed and Sensitivity with Guanidine Chloride. BioTechniques 2020, 69 (3), 178-185; Zhang et al., Improved Visual Detection of DNA Amplification Using Pyridylazophenol Metal Sensing Dyes. Commun. Biol. 2022, 5 (1), 999; and Szobi et al., Vivid COVID-19 LAMP Is an Ultrasensitive, Quadruplexed Test Using LNA-Modified Primers and a Zinc Ion and 5-Br-PAPS Colorimetric Detection System. Commun. Biol. 2023, 6 (1), 233.

However, typical LAMP and RT-LAMP assays generally exhibit a turnaround time (TAT) of about 30 minutes to an hour, and the assay sensitivity and specificity largely depend on assay formulation and primer designs. Various techniques have aimed to accelerate LAMP reactions. See, for example: Nagamine et al., Accelerated Reaction by Loop-Mediated Isothermal Amplification Using Loop Primers. Mol. Cell. Probes 2002, 16 (3), 223-229; Dangerfield et al., Kinetics of Elementary Steps in Loop-Mediated Isothermal Amplification (LAMP) Show That Strand Invasion during Initiation Is Rate-Limiting. Nucleic Acids Res. 2023, 51 (1), 488-499; Gandelman et al., Loop-Mediated Amplification Accelerated by Stem Primers. Int. J. Mol. Sci. 2011, 12 (12), 9108-9124; Martineau et al., Improved Performance of Loop-Mediated Isothermal Amplification Assays via Swarm Priming. Anal. Chem. 2017, 89 (1), 625-632. Despite such efforts, achieving a significant decrease in TAT while maintaining high performance has proven to be challenging.

Accordingly, there remains an ongoing need for improved assays and methods for isothermal amplification and detection of nucleic acids.

### SUMMARY

The present disclosure relates to assay formulations and methods that enable ultra-fast (e.g., 5 to 15 min TAT, depending on target concentration), one-pot detection of nucleic acids with effective sensitivity. The disclosed assay and method can be utilized with different types of real-time or end-point readout methods to provide quantitative, semi-quantitative, or qualitative readout of the test result. Example implementations include an ultra-rapid LAMP or RT-LAMP assay with real-time fluorescent readout (based on intercalating dyes or fluorescently-labeled probes), pH-independent colorimetric readout, and/or pH-dependent colorimetric readout. Other readout methods suitable for LAMP/RT-LAMP assays can additionally or alternatively be used, such as real-time measurement of turbidity.

The ultra-fast assay disclosed herein can include LAMP/RT-LAMP components such as reaction buffers, salts, strand-displacing polymerase, reverse transcriptase, dNTPs, betaine, surfactants, high-performance enzymes, molecular enhancers, and/or one or more pairs of accelerating primers complementing the 4-6 primer configuration of conventional LAMP/RT-LAMP reactions. A commercially available master mix, such as those disclosed herein and/or others known in the art, can include several of such components, and may contain additional components not listed in the foregoing.

The disclosed ultra-fast assay can be carried out in one-pot format in either solution form (i.e., liquid form reagents and reaction mix), lyophilized form (one-pot freeze-dried reaction mix), or air-dried form (one-pot air-dried reaction mix).

The examples disclosed herein demonstrate rapid and sensitive detection of target viruses or nucleic acids by three types of assay readouts (fluorescent, pH-independent colorimetric, and pH-dependent colorimetric). The disclosed assays can also be formulated for both liquid form and one-pot stabilized form (e.g., reaction mixture lyophilized into a room-temperature-stable pellet, such as a lyobead or lyosphere).

The disclosed ultra-fast assays can exhibit benefits over conventional assays, such as rapid TAT, simplified workflow, and expanded temperature tolerance ranges compared to conventional LAMP/RT-LAMP. Quantification of the target nucleic acid can also be carried out by analyzing fluorescence resulting from assays that include a fluorescent readout.

The disclosed assays can also provide ultra-rapid, sensitive, and specific detection of various targets by simply switching the primer sets.

The disclosed assays can provide effective speed, dynamic range, and limit of detection (LoD), and have the capability for real-time quantification of different targets, such as when using the disclosed chemistry with fluorescent readout.

The ultra-fast assay (e.g., an embodiment with colorimetric readout) can be integrated into a low-cost, disposable, electricity-free chemical heating assay device for the sensitive molecular detection of respiratory viruses, enabling a simple, streamlined workflow from swab to result without need for instrumentation.

The disclosed assays and related methods can integrate sample processing (including but not limited to sample inactivation, sample lysis, nucleases inhibition, nucleic acid extraction, nucleic acid stabilization), nucleic acid amplification, and result readout into a single, ultra-fast one-pot reaction, thus enabling the molecular assay to be easily completed with minimal steps inside a single reaction vessel. Alternatively, the disclosed assay chemistries can also be used in combination with any suitable nucleic acid extraction, purification, and/or concentration protocols known in the art to further improve performance.

The disclosed ultra-fast assay can enable rapid detection of different types of targets from different types of samples. The sample can be a lower nasal swab sample, nasopharyngeal swab sample, gingival swab sample, buccal swab sample, gargle sample, sputum sample, and/or saliva sample. The sample can additionally or alternatively include other bodily fluids depending on the type of disease, biomarker, and/or pathogen being targeted. The sample can include an environmental sample collected from soil or water, for example. The sample can include agricultural or food products such as feedstocks, vegetables, fruits, meat, milk, honey, and the like. The target of the molecular diagnostic test may include a virus, bacteria, algae, or other type of microorganism.

The ultra-fast assay presented herein can be conducted using any suitable heating device, such as a heating device operated with electricity (e.g., USB or battery-powered) or without electricity (e.g., chemical heating devices such as those based on exothermic reactions and optionally with phase change materials), that can effectively provide substantially constant temperature for the LAMP or RT-LAMP reaction (e.g., 65° C. or a specific temperature or a temperature range optimized based on the design of primers and assay formulation).

The ultra-fast assay described herein can significantly improve the cost-efficiency, throughput, and performance of molecular testing across various settings, including low-cost at-home molecular tests, professional molecular tests at the point of care (POC) (e.g., hospital, doctor's office, walk-in clinic), and high-throughput molecular tests (e.g., lab-developed tests).

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Various objects, features, characteristics, and advantages of the disclosure will become apparent and more readily appreciated from the following description, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIGS. 1A and 1B show performance of an ultra-fast, one-pot RT-LAMP assay formulated in both liquid form (FIG. 1A) and one-pot lyophilized (lyobead) form (FIG. 1B).

FIGS. 2A-2C show photos of one-pot lyophilized assay lyobeads designed for detection of different respiratory pathogens (SARS-CoV-2, Flu A, Flu B, RSV) and positive control (human RNaseP and 18 S) from nasal swab samples. Examples of lyobead performance after 209 days of accelerated thermal stability testing at 45° C. (FIGS. 2B and 2C) illustrate the stability and robustness of the assay lyobeads.

FIG. 3 shows the performance of the ultra-fast, one-pot lyophilized RT-LAMP assay integrated into a low-cost, disposable assay device (Domus At-Home Test, available from Domus Diagnostics, Inc.) powered by electricity-free chemical heating. In this example, various concentrations of SARS-CoV-2 Omicron virus were prepared in nasal wash, with 50 μL spiked onto swab. The swab was then eluted into the lysis buffer tube of the device, and the assay was performed according to the instructions for use (IFU). Preliminary LoD was determined to be 0.4-0.9 virus copies per microliter in the final reaction on the assay device.

FIGS. 4A and 4B show temperature tolerance testing of a COVID assay implemented with a slower, conventional RT-LAMP formulation. COVID samples consist of SARS-CoV-2 virus diluted in lysis buffer with RNaseA added to simulate the effect of nucleases from nasal swabs. According to the results, the conventional assay is incompatible with temperatures of 70° C. or higher or 62° C. and lower, and the effective temperature range of the assay is determined to be 64-68° C.

FIGS. 5A and 5B show temperature tolerance testing of a COVID assay implemented with the ultra-fast, one-pot RT-LAMP formulation. COVID samples consist of SARS-CoV-2 virus diluted in lysis buffer with RNaseA added to simulate the effect of nucleases from nasal swabs. The results indicate that the effective temperature range for a 15-minute turnaround time (TAT) assay is extended to 55-70° C. and for a 10-minute turnaround time (TAT) assay is extended to 60-70° C.

FIGS. 6A-6D show results of studies evaluating the speed, dynamic range, and limit of detection of the ultra-fast, one-pot fluorescent RT-LAMP assay formulated in this example for rapid detection and quantification of SARS-CoV-2. Various concentrations of SARS-CoV-2 RNA were detected with high repeatability, demonstrating the assay's sensitivity, specificity, and real-time quantification capability across a broad linear range.

FIGS. 7A-8B show results of studies characterizing the performance of the ultra-fast, one-pot fluorescent RT-LAMP assay formulated in this example for rapid detection and quantification of Flu A and respiratory syncytial virus (RSV). Various concentrations of Flu A and RSV RNA were detected with high repeatability, demonstrating the assay's sensitivity, specificity, and quantification capability across broad linear ranges for different targets.

FIGS. 9A and 9B show examples of amplification curve analyses from the ultra-fast, one-pot fluorescent RT-LAMP assay. Tailored LAMP kinetic models and real-time feature extractions are applied to determine the assay's time to positive (TTP), enabling reliable positive detection, real-time quantification, and differentiation of positives from negatives with short overall turnaround time.

FIGS. 10A-10D show examples of the ultra-fast, one-pot fluorescent RT-LAMP and LAMP assay formulated for detecting various targets by simply changing the respective primer sets.

### DETAILED DESCRIPTION

### Overview

The disclosed assay can be used in combination with any suitable nucleic acid extraction, purification, and/or concentration protocols known in the art. In some embodiments, to reduce cost and complexity of molecular testing workflows, the assay can integrate sample processing (including but not limited to sample inactivation, sample lysis, nucleases inhibition, nucleic acid extraction, nucleic acid stabilization), nucleic acid amplification, and result readout into a single, ultra-fast one-pot reaction, thus enabling the assay to be easily completed with minimal steps inside a single reaction vessel.

Applications of the disclosed assay include the detection of pathogens, diagnostics of cancers and infectious diseases, SNP genotyping, quality control of food and dietary products, and environmental surveillance, for example.

The disclosed assay can include or incorporate features described in: International Application No. PCT/US2022/021138, published as WO 2022/204023; U.S. application Ser. No. 17/749,858, published as US 2022/0372569; and/or U.S. application Ser. No. 18/116,138, published as US 2023/0279478, each of which is hereby incorporated by reference in its entirety.

The disclosed assay can include components described in US Patent Publication No. 2024/0093278-A1, which is incorporated herein by reference in its entirety. For example, the presently disclosed assay can include the buffer and/or master mix components described therein (see, e.g., paragraphs [0039]-[0047]), can include the readout indicator components described therein (see, e.g., paragraphs [0048]-[0051]), and/or can be implemented using the one-pot methods and/or kits described therein (see paragraphs [0022]-[0035]).

The disclosed assay can be associated with and/or carried out using an assay device such as disclosed in US Patent Publication No. 2023/0392063-A1, U.S. Provisional Patent Application No. 63/660,882, U.S. patent application Ser. No. 18/970,704, each of which is incorporated herein by reference in its entirety.

Throughout this disclosure, certain examples are described in the context of RNA as the target nucleic acid. It will be understood that similar components and method steps may be readily utilized for assays targeting DNA with minimal modification (e.g., removal of reverse transcriptase). The converse is also true. That is, example assays describing DNA as the target nucleic acid can be readily adapted to target RNA with minimal modification (e.g., the addition of a suitable reverse transcriptase and/or the use of a polymerase with sufficient reverse transcriptase activity).

Similarly, examples describing RT-LAMP can also be adapted to LAMP, and vice versa, as appropriate for the type of nucleic acid targeted by the assay. Thus, references to LAMP or RT-LAMP should be understood as applicable to both.

### Example Master Mix Formulations

The master mix may be formulated for fluorescent, pH-dependent colorimetric, or pH-independent colorimetric detection of the target nucleic acid.

The master mix may comprise one or more of the following components (with recited concentrations representing examples only): 4 mM to 8 mM MgSO₄ and/or MgCl₂; 10 mM (NH4)₂SO₄ and/or (NH4)₂Cl₂; 1 mM to 2 mM dNTP mix; DNA polymerase (e.g., 0.32 U/μL Bst 2.0 or Bst 2.0 WarmStart® DNA Polymerase); 0.1% to 2% Tween 20 and/or Triton-100 (e.g., pH 8.8); 2 mM to 20 mM Tris-HCl (pH 8.8) and/or Tris or TE at suitable concentration; 10 mM to 50 mM KCl; 0.8 mM betaine; 10 mM to 60 mM GuHCl; and/or thermolabile Uracil-DNA-glycosylase (UDG) and dUTP to prevent carryover contamination.

Suitable commercially available LAMP and RT-LAMP master mixes include WarmStart® Multi-Purpose LAMP/RT-LAMP 2× Master Mix with UDG (New England Biolabs M1708), WarmStart® Colorimetric LAMP 2× Master Mix with UDG (New England Biolabs M1804), SuperScript IV RT-LAMP Master Mix (Invitrogen A51802), and their lyo-ready, glycerol-free, and/or high-concentration versions.

The master mix can optionally include a second DNA polymerase in addition to a first DNA polymerase such as described above. The second DNA polymerase can include a strand-displacing DNA polymerase such as Lyo-ready Bst DNA Polymerase (polymerase based on the large fragment of Bst DNA polymerase) (sold by Invitrogen, A56656) or its related versions (e.g., standard, lyo-ready, glycerol-free, and/or high-concentration).

For RT-LAMP applications, the master mix may include (e.g., at 0.2 U/μL) a reverse transcriptase, such as WarmStart® RTx Reverse Transcriptase (New England Biolabs). The master mix can additionally include a second reverse transcriptase such as Lyo-ready SuperScript IV Reverse Transcriptase (reverse transcriptase based on of the Moloney Murine Leukemia Virus (M-MLV) reverse transcriptase) (sold by Invitrogen, EP164B1B008) or its related versions (e.g., standard, lyo-ready, glycerol-free, and/or high-concentration).

According to application needs, the master mix may further comprise one or more additives known in the art to enhance the performance (e.g., sensitivity, specificity, speed, robustness) of nucleic acid amplification reactions, including but not limited to: crowding agents (e.g., polyethylene glycol (PEG), Ficoll, dextran), dsDNA destabilizers (e.g., helicase, recombinases, endonucleases, ionic liquids, proline), dsDNA stabilizers (e.g., Tetramethylammonium chloride (TMAC)), enzyme stabilizers (e.g., bovine serum albumin (BSA), trehalose, pullulan), template blockers (e.g., single-stranded DNA-binding proteins (SSBs), graphene oxide (GO), cobalt oxyhydroxide (CoOOH) nanoflakes), and/or oligonucleotide modifications or analogs (e.g., locked nucleic acids (LNA), and/or phosphorothioate nucleotide analogues (PTO), peptide nucleic acids (PNA)).

The master mix can be formulated in liquid solution form, freeze-dried (i.e., lyophilized) form, or air-dried form.

The master mix may include one or more excipients (including but not limited to sucrose, trehalose, dextran, pullulan, lactose, glucose, raffinose, mannitol, sorbitol, glycine, histidine, arginine, gelatin, dextrose, hydroxyethyl starch, poly(ethylene glycol), poly(propylene glycol), poly(vinyl alcohol), polyvinylpyrrolidone (PVP), or combinations thereof) to form stabilized reaction mixes (e.g., by techniques such as air drying or lyophilization) to enable extended storage at ambient conditions. For example, the master mix may be formulated for lyophilization and may include trehalose (5% to 20% w/v, such as 10% w/v) and dextran (2% to 20% w/v, such as 3% to 10% w/v, such as 4% w/v), and can be controlled to a pH of 8.1.

### Example Primers

Primers for carrying out the amplification reaction can be included in the master mix and/or can be separate from the master mix. A “primer set” or “set of primers” refers to a set of primers that function together to enable amplification of a specific corresponding target nucleic acid. The disclosed assay can include a single primer set or can include multiple primer sets. Where multiple primer sets are included, multiple primer sets can be formulated to target different regions of the same sample target (e.g., different regions of the same target microorganism/pathogen). Additionally, or alternatively, the assay can target different sample targets (e.g., different microorganisms/pathogens), where one or more primer sets each target a different sample target.

Often, an end user will add one or more sets of primers, each set designed according to a specific nucleic acid to be targeted by the assay. Primer sequence design is within the capabilities of the skilled person. LAMP or RT-LAMP primer design could be carried out, for example, using known LAMP/RT-LAMP primer design tools such as PrimerExplorer, NEB LAMP Primer Design Tool, or other equivalent software.

The LAMP/RT-LAMP primers can include (e.g., for each primer set) forward outer primer (i.e., F3 primer), backward outer primer (i.e., B3 primer), forward inner primer (i.e., FIP primer), and backward inner primer (i.e., BIP primer) formulated for specific amplification of the corresponding target nucleic acid sequence of the sample. The LAMP/RT-LAMP primers can also include, independently for each primer set, one or more loop primers (i.e., LoopF primer and/or LoopB primer). The LAMP/RT-LAMP primers can also include, independently for each primer set, one or more accelerating primers. The accelerating primers can include stem primers, swarm primers, and/or other primers with similar functional mechanisms. The inclusion of one or more accelerating primers can beneficially accelerate the LAMP/RT-LAMP reaction and/or improve the performance of the reaction (e.g., sensitivity or repeatability at lower detection limit).

LAMP/RT-LAMP primers can be included in the final reaction at, for example, 0.8 μM F3 primer, 0.8 μM B3 primer, 1.6 μM FIP primer, 1.6 μM BIP primer, and when included, 0.4-0.8 μM LoopF primer and/or 0.4-0.8 μM LoopB primer, and optionally, one or more accelerating primers at proper concentrations (e.g., 3.2 μM swarm primers).

### Example Readout Indicators

The readout indicator can be formulated to function according to pH, turbidity, fluorescence, nanomaterials (e.g., gold nanorods/nanoparticles), detection of pyrophosphate, detection of metal ions, a lateral flow strip/dipstick mechanism, gel/capillary electrophoresis, microfluidics, microarrays, electrochemical sensors, molecular transducers, or a combination thereof. The readout indicator can function to provide quantitative, semi-quantitively, or qualitative readout for the reaction. The readout indicator may be included in the sample buffer and/or the master mix according to, for example, stability requirements and/or other application parameters. The readout indicator can be omitted in cases where the reaction can be monitored by, for example, real-time turbidity measurement and/or measurement using electrochemical sensors.

The readout indicator can include a fluorescent double-stranded DNA intercalating dye (e.g., at 1-2× final concentration) such as commercially available dye such as EvaGreen® (Biotium), EvaGreen® Plus (Biotium), dsGreen (Lumiprobe), SYBR® Green I, and/or SYTO™ 9 (Invitrogen). The readout indicator can include a colorimetric double-stranded DNA intercalating dye (e.g., at 0.004% w/v) such as leuco crystal violet, malachite green, and/or methyl green.

The readout indicator can include a sequence-specific probe-based fluorescence-generating mechanism, such as probes with fluorophore-quencher pairs. Examples include molecular beacons, Scorpions probes, strand-displacement probes such as assimilating probes, detection of amplification by releasing of quenching (DARQ) probes, and/or oligonucleotide strand exchange (OSD) probes.

The readout indicator can include a pH-dependent colorimetric indicator (e.g., 50 μM to 100 μM) such as phenol red, neutral red, cresol red, cresol purple, thymol blue, bromothymol blue, bromophenol blue, litmus, chlorophenol red, dichlorofluorescein, methyl red, bromocresol purple, naphtholphthalein, and/or cresolphthalein.

The readout indicator can include a metal indicator that senses metal ions such as Mg²⁺, Mn²⁺, Zn²⁺, Cu²⁺, Co²⁺, Cd²⁺, Fe²⁺, Ni²⁺, Hg²⁺, Pb²⁺. For example, the readout indicator can include one or more of hydroxynaphthol blue, eriochrome black T, calcein, and/or a pyridylazophenol dye such as 2-(5-Bromo-2-pyridylazo)-5-[N-propyl-N-(3-sulfopropyl)amino]phenol (5-Bromo-PAPS) or 2-(5-Nitro-2-pyridylazo)-5-[N-n-propyl-N-(3-sulfopropyl)amino]phenol (5-Nitro-PAPS). An example assay can include 0.12 mM hydroxynaphthol blue with 25 μM calcein and 0.5 mM MnCl₂. Another example can include 50 μM to 200 μM 5-Bromo-PAPS with 50 μM to 300 μM Mn²⁺.

### Example Sample Buffer Formulations

The sample buffer is formulated to receive a sample that includes or is suspected of including a target microorganism (e.g., virus, bacteria) or nucleic acid (e.g., DNA, RNA). The sample buffer may include one or more components to enable sample elution, inactivation, lysis, nucleases inhibition, nucleic acid extraction, and/or stabilization.

The sample buffer may include, for example: nuclease-free water; a surfactant such as polysorbate 20 (trade name: Tween 20), polysorbate 80 (trade name: Tween 80), polyethylene glycol p-(1,1,3,3-tetramethylbutyl)-phenyl ether (trade name: Triton X-100), 1,1,3,3-Tetramethylbutyl)phenyl-polyethylene glycol, (trade name: Triton X-114), octylphenoxypolyethoxyethanol (trade name: Nonidet P-40 or NP-40), branched octylphenoxypolyethyleneoxyethanol (trade name: Igepal CA-630), 3-[(3-cholamidopropyl)dimethylammonio]-1-propanesulfonate (CHAPS), and/or sodium dodecyl sulfate (SDS); a reducing/denaturing agent such as dithiothreitol (DTT), tris(2-carboxyethyl)phosphine (TCEP), urea, guanidine hydrochloride (GuHCl), guanidinium thiocyanate (GITC), and/or formamide; a nuclease inhibitor such as proteinase K, murine RNase inhibitor, human placenta RNase inhibitor, vinylsulfonic acid (VSA), polyvinylsulfonic acid (PVSA), achromopeptidase (ACP), native or recombinant nuclease inhibitors such as those sold under the trade names RNasin Ribonuclease Inhibitor, RNasin Plus Ribonuclease Inhibitor, RiboLock RNase Inhibitor, SUPERase•In, RNaseOUT, and/or RNAsecure. The sample buffer may further comprise one or more of: chelating agents such as ethylenediaminetetraacetic acid (EDTA); and/or buffering salts such as tris(hydroxymethyl)aminomethane (commonly referred to as Tris), Tris-HCl, Tris-EDTA (TE), Tris-acetate-EDTA (TAE), Tris-borate-EDTA (TBE), HCl solution, NaOH or KOH

### Solution

In some embodiments, one or more components typically included in the master mix (e.g., MgSO₄, MgCl₂, colorimetric indicator, etc.) may be additionally or alternatively included in the sample buffer. In some embodiments, one of more components typically included in the sample buffer (e.g., nuclease inhibitor, etc.) may be additionally or alternatively included in the master mix (e.g., in air-dried or lyophilized form) according to cost and/or stability considerations. For example, including one or more nuclease inhibitors in the master mix allows for stabilization via air drying or lyophilization.

In one embodiment, the sample buffer can include the following, optionally included at the recited concentrations: a surfactant such as Tween 20 and/or Triton X-100 at a concentration ranging from about 0.025% to about 10%, or about 0.05% to about 7.5%, or about 0.075% to about 5%, or about 0.1% to about 2.5%, or about 0.1% to about 1%, or a value within a range with endpoints defined by any two of the foregoing values; reducing/denaturing agents including (1) formamide at a concentrations ranging from about 0.1% to 10%, or about 0.5% to about 8.5%, or about 1% to about 7%, or about 2% to about 6%, or a value within a range with endpoints defined by any two of the foregoing values, and/or (2) urea at a concentration ranging from about 0.5 mM to 1.8 M, or about 1 mM to 1M, or about 2 mM to 250 mM, or about 2.5 mM to 100 mM, or about 5 mM to 50 mM, or about 7.5 m to 25 mM, or a value within a range with endpoints defined by any two of the foregoing values; nuclease inhibitors including VSA or PVSA at a concentration from about 0.01 mg/mL to 10 mg/mL, or about 0.05 mg/mL to 5 mg/mL, or about 0.1 mg/mL to 1 mg/mL, or a value within a range with endpoints defined by any two of the foregoing values; and/or murine RNase inhibitor or human placenta RNase inhibitor or RNasin Ribonuclease Inhibitor (native or recombinant) or RNasin Plus Ribonuclease Inhibitor or RiboLock RNase Inhibitor or RNaseOUT at a concentration ranging from about 0.05 units/μL to 2 units/μL, or about 0.1 units/μL to 1 units/μL, or a value within a range with endpoints defined by any two of the foregoing values. As discussed above, in some embodiments, one or more nuclease inhibitors may be additionally or alternatively included in the master mix.

The sample buffer can optionally further comprise: a buffering salt such as TBE at a concentration ranging from about 0.001× to about 1×, or about 0.0015× to about 0.5×, or about 0.002× to about 0.25×, or about 0.0025× to about 0.1×, or a value within a range with endpoints defined by any two of the foregoing values; and/or a chelating agent such as EDTA at a concentration ranging from about 0.005 mM to 2.5 mM, or about 0.05 mM to about 1.5 mM, or about 0.5 mM to about 1 mM, or a value within a range with endpoints defined by any two of the foregoing values. In some cases, the sample buffer is adjusted to pH about 8.0-8.2 for optimal compatibility with the nucleic acid amplification master mix.

In some embodiments, the sample buffer comprises 0.1% Tween 20 and 10 mM urea in nuclease-free water, optionally additionally including one or more of the foregoing components. Such a sample buffer may be referred to herein as a “Domus buffer”.

### Example Kits

The present disclosure also relates to a kit for amplifying and detecting a target nucleic acid. The kit can comprise: a sample collection device; a first vessel that contains a sample processing buffer; a second vessel that contains a frozen, air-dried, or lyophilized nucleic acid amplification master mix formulated for nucleic acid amplification and result readout; and a device for transferring a portion of a sample mixture from the first vessel into the second vessel.

The kit can optionally further include a heat source configured to generate heat at a temperature profile suitable for performing a one-pot sample processing and nucleic acid amplification reaction. The kit can optionally further include a reader device to aid in interpretation and report of the test result (e.g., qualitatively, semi-quantitatively, or quantitatively) in a semi- or fully automatic fashion. For example, the kit can include, or be used in conjunction with, the colorimetric assay visualization system described in U.S. patent application Ser. No. 18/752,268, which is incorporated herein by reference in its entirety.

The heat source can be internally powered or externally powered, and may be battery-powered, USB-powered, solar-powered, chemically powered by an exothermic reaction, and variations and combinations thereof. The kit can further comprise a mechanism for regulating the output temperature profile. For example, the kit can include an insulative container configured with sufficient insulation and proper venting to maintain a desired output temperature profile over a period of time for effectively carrying out the unified-one-pot reaction.

### Example Sample Sources

A “sample”, as used herein, is any source that includes or is suspected of including a target microorganism or nucleic acid, whether unprocessed or mixed with one or more receiving or processing buffers such as the processing buffer disclosed herein. The sample can include, for example, DNA and/or RNA extracted from microorganisms (e.g., from viruses, bacteria, algae, or other types of microorganisms) or from other biological samples; processed or unprocessed biological samples; and/or synthetic nucleic acids.

The sample can include bodily fluids relevant to targeted diseases, biomarkers, or pathogens. For example, the sample can include a lower nasal swab sample, nasopharyngeal swab sample, gingival swab sample, buccal swab sample, gargle sample, sputum sample, or saliva sample (e.g., prepared in a suitable buffer).

The sample can include a veterinary sample. The sample can include an environmental sample such as a water and/or soil sample. The sample can include agricultural products or food products such as a feedstocks, vegetable, fruit, meat, milk, honey, etcetera.

### Additional Terms & Definitions

For assays detecting pre-extracted RNA/DNA samples, the term turnaround time (TAT) refers to the time required for the amplification reaction to sufficiently amplify target nucleic acids and activate the readout indicator sufficient to enable the differentiation of positive samples from negative samples, such as a colorimetric readout distinguishing positive from negative results by visual inspection. When the sample is an unprocessed biological sample, such as a nasal swab containing mucus, nucleases, and respiratory viruses, the TAT additionally includes the sample preparation time, and thus equals the total time from mixing of the unprocessed sample into the preparation buffer (e.g., swab elution into the buffer) to the final result readout. The disclosed ultra-fast one-pot LAMP/RT-LAMP chemistry can be used in conjunction with the lysis buffer disclosed herein, in this approach enabling one or more of sample elution, dilution, inactivation, lysis, nucleic acid extraction, and stabilization, which can beneficially reduce the extraction portion of the process. The term “time to positive” (TTP) refers to the determination and differentiation of positive signals from negative signals in real-time fluorescent LAMP/RT-LAMP assays, starting at the beginning of the isothermal reaction, based on the analysis of amplification curves.

While certain embodiments of the present disclosure have been described in detail, with reference to specific configurations, parameters, components, elements, etcetera, the descriptions are illustrative and are not to be construed as limiting the scope of the claimed invention.

Concentration and amount values for the components disclosed herein are examples, and other embodiments may include different concentrations and/or amounts of one or more of the components. For example, disclosed concentration and/or amount values (including range endpoints) may be independently adjusted by ±20%, ±15%, ±10%, ±5%, or ±1% from the displayed values. When concentrations are given, they should be understood to represent the final concentration for the amplification reaction, unless indicated otherwise. Concentration amounts expressed as percentages shall be taken to mean % w/v unless indicated otherwise.

For any given element of component of a described embodiment, any of the possible alternatives listed for that element or component may be used individually or in combination with one another, unless implicitly or explicitly stated otherwise.

The various features of a given embodiment can be combined with and/or incorporated into other embodiments disclosed herein. Thus, disclosure of certain features relative to a specific embodiment of the present disclosure should not be construed as limiting application or inclusion of said features to the specific embodiment. Rather, it will be appreciated that other embodiments can also include such features.

When the terms “about,” “approximately,” “substantially,” or the like are used in conjunction with a stated amount, value, or property/condition, it may be taken to mean an amount, value or condition that deviates by less than 10%, less than 5%, less than 1%, less than 0.1%, or less than 0.01% of the stated amount, value, or condition. Each numerical parameter should be construed in light of the number of reported significant digits and by applying ordinary rounding techniques.

Any headings and subheadings used herein are for organizational purposes only and are not meant to be used to limit the scope of the description or the claims.

As used in this specification and the appended claims, the singular forms “a,” “an” and “the” do not exclude plural referents unless the context clearly dictates otherwise. Thus, for example, an embodiment referencing a singular referent may also include two or more such referents.

The embodiments disclosed herein should be understood as comprising/including disclosed components, and may therefore include additional components not specifically described. Optionally, the embodiments disclosed herein are essentially free or completely free of components that are not specifically described. That is, non-disclosed components may optionally be completely omitted or essentially omitted from the disclosed embodiments. For example, master mix components, processing buffer components, and/or primer types, not specifically disclosed herein are optionally essentially or completely omitted.

An embodiment that “essentially omits” or is “essentially free of” a component may include trace amounts and/or non-functional amounts of the component. For example, an “essentially omitted” component may be included in an amount no more than 1%, no more than 0.5%, no more than 0.1%, or no more than 0.01% by total weight of the composition.

A composition that “completely omits” or is “completely free of” a component does not include a detectable amount of the component (i.e., does not include an amount above any inherent background signal associated with the testing instrument) when analyzed using standard compositional analysis techniques such as, for example, chromatographic techniques (e.g., thin-layer chromatography (TLC), gas chromatography (GC), liquid chromatography (LC)), or spectroscopy techniques (e.g., Fourier transform infrared (FTIR) spectroscopy).

### Working Examples

The following assays were carried out to detect various targets using various readout methods, including real-time fluorescent readout, pH-independent colorimetric readout, and pH-dependent colorimetric readout.

The skilled person will understand that the components used in these working examples are examples only and can be substituted in whole or in part with alternatives known to provide the same function. Thus, for example, the specific DNA polymerases, reverse transcriptase enzymes, excipients, commercial master mixes, etc. used in these examples can be substituted in whole or in part with alternative components that can provide substantially similar function.

Moreover, the specific amounts and concentrations used in these examples can be adjusted without detrimentally affecting the results. For example, in at least some cases, disclosed concentration and/or amount values may be independently adjusted by ±20%, ±15%, ±10%, ±5%, or ±1% from the displayed values. The skilled person is readily able to run amplification reaction testing to determine functional ranges of the disclosed components.

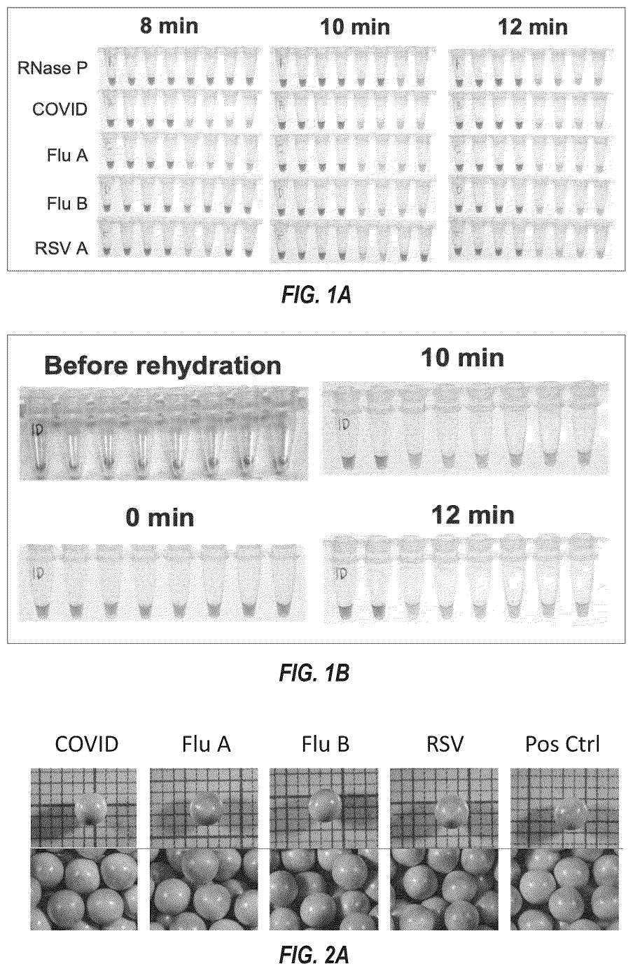

The assays were carried out with primer sets designed to target the respective target pathogens as described below. Each primer set included final reaction concentrations of 0.2 μM for F3 and B3 primers, 1.6 μM for FIP and BIP primers, and 0.4 μM for LoopF and LoopB primers (except for the RNaseP primer set, which included LoopF and LoopB primers at 0.8 μM). For the examples in which SARS-CoV-2 was the target, the primers included primer sets targeting the E1, N2, and Orf1a regions. In the testing related to FIG. 1B, accelerating primers (swarm primers) targeting the N2 region were further included at 3.2 μM each. The skilled person is readily capable of designing primer sets specific for a desired target.

### Example 1: pH-independent Colorimetric Readout

An ultra-fast one-pot RT-LAMP assay was carried out to detect multiple different targets (RNase P, SARS-CoV-2, Flu A, Flu B, RSV A) using a pH-independent colorimetric readout in liquid form (FIG. 1A) and lyophilized (lyobead) form (FIG. 1B). The components used in the assay are shown in Table 1. Total reaction volume was 15 μL for the liquid form and following resuspension of the lyobead form.

TABLE 1
Assay with pH-Independent Colorimetric Readout

| | Final Reaction | |
|---|---|---|
| Component | Concentration | |
| Primers (designed independently for each | (see above) | |
| respective target) | | |
| WarmStart ® Multi-Purpose LAMP/RT-LAMP 2X | 1X | |
| Master Mix with UDG (NEB M1708S)* | | |
| Lyo-Ready Bst DNA Polymerase (Invitrogen ™ | 0.268 | U/μL |
| A56656 or A56657) | | |
| Lyo-Ready SuperScript ™ IV Reverse | 0.333 | U/μL |
| Transcriptase (Invitrogen ™ EP164B1B008 or | | |
| EP164B2B014) | | |
| GuHCl | 40 | mM |
| RNase inhibitor (RNasin Plus)** | 0.1 | U/μL |
| 5-Bromo-PAPS | 100 | μM |
| MnCl₂ | 175 | μM |
| Trehalose*** | 5% | w/v |

*Includes Bst 2.0 WarmStart ® DNA Polymerase, WarmStart RTx Reverse Transcriptase, dUTP, and thermolabile UDG in a buffer solution. Use glycerol-free, lyo-ready, or high-concentration alternative for assays requiring lyophilization. An example alternative is WarmStart ® Multi-Purpose LAMP/RT-LAMP 4X Master Mix with UDG (NEB M1718B).
**For receiving samples containing nucleases. Can optionally be included up to 2 U/μL or more as needed. Alternative formulation can be RNaseOUT ™ (Invitrogen ™ EO2521B001 or EO2522B001) at 0.1-2 U/μL or more final concentration.
***For lyophilized assays, bring total volume to bead droplet size (16.5 μL in these examples) while achieving desired excipient final concentration. Excipient optionally included up to 10% w/v or more as needed. Alternative excipient formulation can be 10% Trehalose + 4% Dextran (pH 8.1). May also be included in liquid assays (as is the case for the liquid assays of these examples). For liquid assays, nuclease-free water may be added so that the total reaction volume after adding sample (RNA in nuclease-free water or virus in Domus buffer) is 15 μL. For lyobead assays, rehydration volume of 16.5 μL bead is 15 μL.

The results for the liquid samples are shown in FIG. 1A. The reaction tubes were arranged with non-template controls (NTC) and spiked samples according to (from left to right):
RNase P: NTC×4, nasal swab×4
SARS-CoV-2: NTC×4, 666 cp/μL×2, 66 cp/μL×2
Flu A: NTC×4, 0.0004 ng/μL×2, 0.0002 ng/μL×2
Flu B: NTC×4, 0.000004 ng/μL×2, 0.000002 ng/μL×2
RSV A: NTC×4, 0.0506 ng/μL×2, 0.00506 ng/μL×2
   The reactions were incubated at a constant temperature of 65° C. The reactions were chilled on ice for 1 minutes to stabilize the colors before the photos were taken.

The results following resuspension of the lyophilized SARS-CoV-2 samples are shown in FIG. 1B. The lyobeads were resuspended in water spiked with SARS-CoV-2 RNA. Each strip of 8 samples included (from left to right) NTC×2, 8.3 cp/μL×3, and 4.16 cp/μL×3.

Positive detection is indicated by a change from pink to yellow, shown as change from dark to light in grayscale images. For the liquid samples, all targets exhibited positive results within 12 minutes (SARS-CoV-2 and Flu A at 8 minutes, Flu B at 10 minutes, RSV and RNase P at 12 minutes) with no false positives. For the lyophilized samples, all targets exhibited positive results within 10 minutes with no false positives. The results indicated effective detection in a short timeframe for both liquid and lyophilized form. Additional testing discussed below further illustrates the effectiveness and benefits of the ultra-fast one-pot assay.

### Example 2: pH-Dependent Colorimetric Readout

An ultra-fast one-pot RT-LAMP assay was carried out to detect multiple different samples of SARS-CoV-2 RNA using a pH-dependent colorimetric readout. The components used in the assay are shown in Table 2. Total reaction volume was 15 μL.

TABLE 2
Assay with pH-Dependent Colorimetric Readout

| | Final Reaction | |
|---|---|---|
| Component | Concentration | |
| Primers | (see above) | |
| WarmStart ® Colorimetric LAMP 2X Master Mix | 1X | |
| with UDG (NEB M1804S)* | | |
| Lyo-Ready Bst DNA Polymerase (Invitrogen ™ | 0.268 | U/μL |
| A56656 or A56657) | | |
| Lyo-Ready SuperScript ™ IV Reverse | 0.333 | U/μL |
| Transcriptase (Invitrogen ™ EP164B1B008 or | | |
| EP164B2B014) | | |
| GuHCl | 40 | mM |
| RNase inhibitor (RNasin Plus)** | 0.1 | U/μL |
| Trehalose*** | 5% | w/v |

*Includes Bst 2.0 WarmStart ® DNA Polymerase, WarmStart RTx Reverse Transcriptase, dUTP, thermolabile UDG, and phenol red pH indicator in a weakly buffered solution. Use glycerol-free, lyo-ready, or high-concentration alternative for assays requiring lyophilization.
**For receiving samples containing nucleases. Can optionally be included up to 2 U/μL as needed. Alternative formulation can be RNaseOUT ™ (Invitrogen ™ EO2521B001 or EO2522B001) at 0.1-2 U/μL or more final concentration.
***For lyophilized assays, bring total volume to bead droplet size (16.5 μL in these examples) while achieving desired excipient final concentration. Excipient optionally included up to 10% w/v or more as needed. Alternative excipient formulation can be 10% Trehalose + 4% Dextran (pH 8.1). May also be included in liquid assays (as is the case for the liquid assays of these examples). For liquid assays, nuclease-free water may be added so that the total reaction volume after adding sample (RNA in nuclease-free water or virus in Domus buffer) is 15 μL. For lyobead assays, rehydration volume of 16.5 μL bead is 15 μL.

The samples included multiple replicates of NTC, and multiple replicates of positive samples spiked with various concentrations of SARS-CoV-2 RNA. The reactions were incubated at a constant temperature of 65° C. for up to 35 minutes, with reaction colors recorded by a cellphone at various time points. At each time point, the reactions were chilled on ice for 30 seconds to stabilize the colors before the photos were taken. Positive detection is indicated by a change from pink to yellow. According to the results (not shown), successful and consistent detection of positive samples down to 66 cp/uL were achieved by 10 minutes, and successful and consistent detection of positive samples down to 3.3 cp/uL were achieved by 12 minutes. Successful detection down to 0.8-1.6 cp/uL was achieved by 14-16 minutes.

### Example 3: Effectiveness & Robustness of Lyobeads

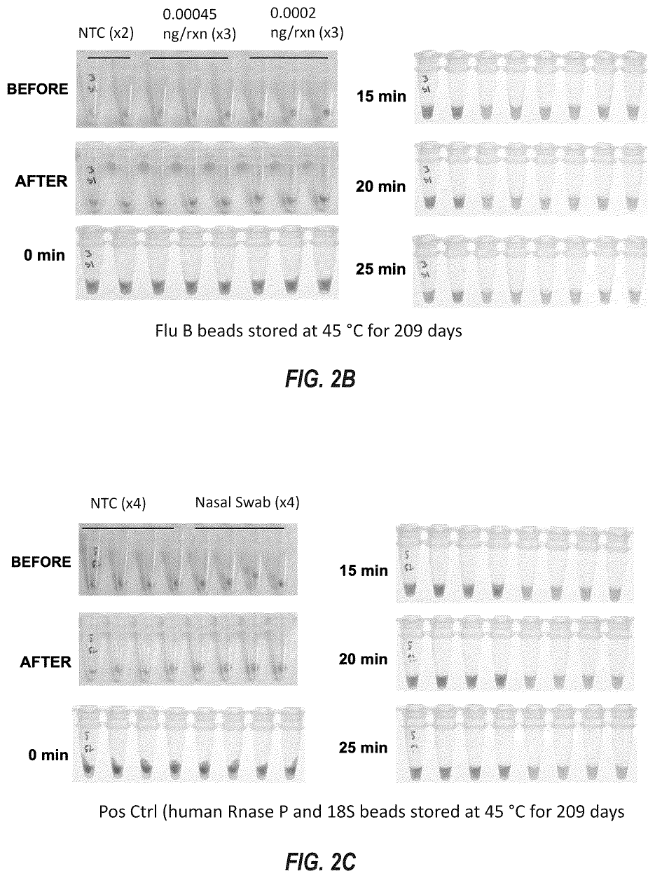

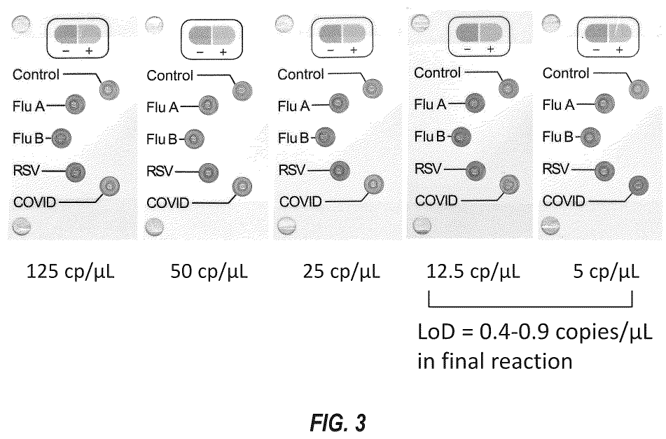

A selection of lyobeads formed according to Example 1 (FIG. 2A) were stored at 45° C. for 209 days before resuspension to the 15 μL reaction volume. Results for assays targeting Flu B and a positive control (human RNase P and 18 S) are illustrated in FIGS. 2B and 2C. The accurate and effective results illustrate the stability and robustness of the assay lyobeads.

### Example 4: Integration with Low-Cost Assay Device

The ultra-fast, one-pot lyophilized assay according to Example 1 was integrated into a low-cost, disposable assay device (Domus At-Home Test, available from Domus Diagnostics, Inc.) powered by electricity-free chemical heating. Various concentrations (as illustrated) of SARS-CoV-2 Omicron virus were prepared in nasal wash, with 50 μL spiked onto swab. The swab was then eluted into the lysis buffer tube of the device, and the assay was performed according to the instructions for use (IFU). Preliminary LoD was determined to be 0.4-0.9 virus copies per microliter in the final reaction on the assay device.

### Example 5: Comparison to Conventional RT-LAMP Assay

As a comparison example, a conventional RT-LAMP chemistry was formulated as shown in Table 3.

TABLE 3
Conventional RT-LAMP Assay Chemistry

| | Final Reaction |
|---|---|
| Component | Concentration |
| Primers | Same as Table 1 |
| WarmStart ® Multi-Purpose LAMP/RT-LAMP 2X | Same as Table 1 |
| Master Mix with UDG (NEB M1708S)* | |
| GuHCl | Same as Table 1 |
| RNase inhibitor (RNasin Plus) | Same as Table 1 |
| 5-Bromo-PAPS | Same as Table 1 |
| MnCl₂ | Same as Table 1 |
| Trehalose | Same as Table 1 |
| Nuclease-free water | Same as Table 1 (to |
| | bring to 15 μL) |

*Includes Bst 2.0 WarmStart ® DNA Polymerase, WarmStart RTx Reverse Transcriptase, dUTP, and thermolabile UDG in a buffer solution.

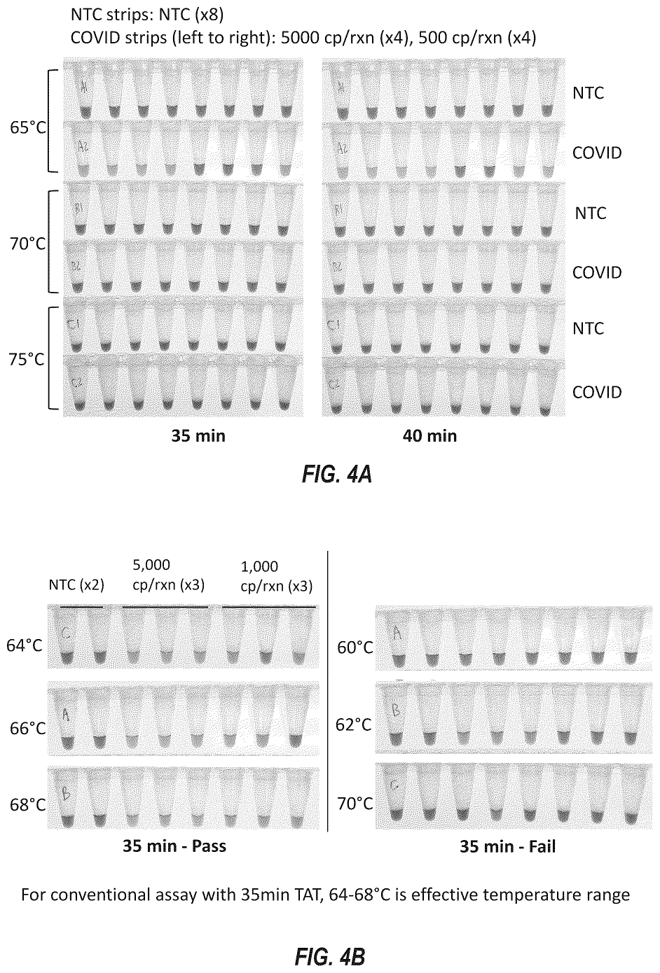

The conventional RT-LAMP chemistry did not include a second Bst DNA Polymerase (the Lyo-Ready Bst DNA Polymerase of Example 1) or a second reverse transcriptase (the SuperScript™ IV Reverse Transcriptase of Example 1), but was otherwise the same as used for the Example 1 version of the ultra-fast RT-LAMP chemistry. The conventional formulation was spiked with various amounts of SARS-CoV-2 virus diluted in Domus buffer with RNaseA added to simulate the effect of nucleases from nasal swabs. The conventional RT-LAMP reaction was carried out at various temperatures, as shown in FIGS. 4A and 4B.

FIG. 4A shows that the conventional assay is incompatible with temperatures of 70° C. or higher. FIG. 4B further shows that the conventional assay is incompatible with temperatures of 62° C. or lower. For a conventional assay with 35-minute TAT, the effective temperature range was determined to be 64-68° C.

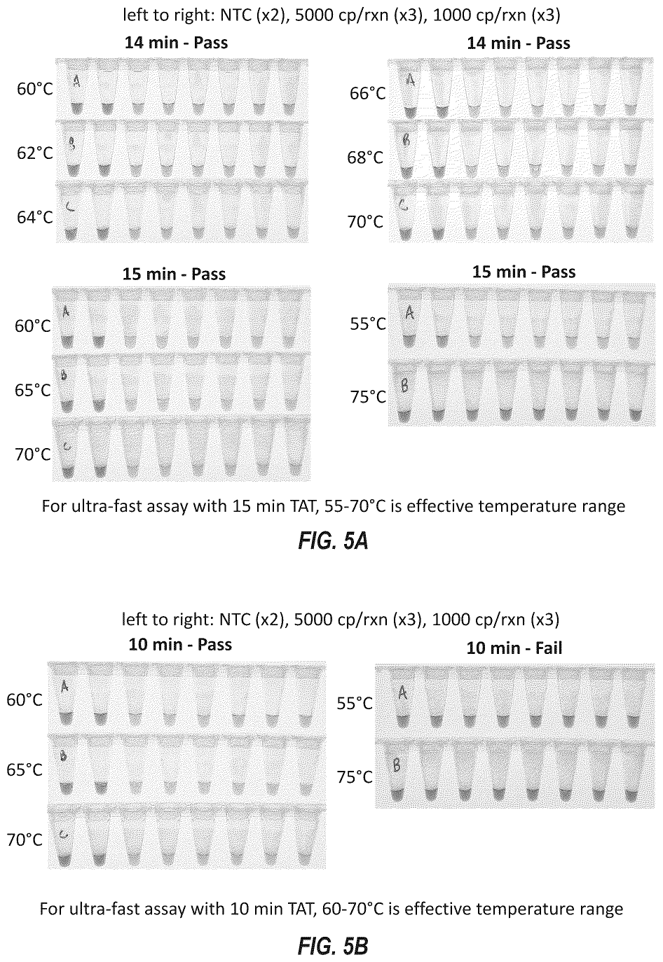

Additional RT-LAMP reactions were carried out using the ultra-fast, one-pot assay of Example 1. As with the assays of FIGS. 4A and 4B, samples were spiked with SARS-CoV-2 virus diluted in Domus buffer with RNaseA added to simulate the effect of nucleases from nasal swabs. FIG. 5A shows that the effective temperature range for a 15-minute TAT is broadened relative to the conventional assay to 55-70° C. FIG. 5B shows that the effective temperature range for a 10-minute turnaround time (TAT) assay is broadened relative to the conventional assay to 60-70° C. The ultra-fast, one-pot assay is therefore significantly faster than the conventional assay and workable within a broader temperature range. Both benefits are advantageous in, for example, remote and/or low-cost applications.

Similar testing was carried out to compare working temperature ranges for other targets. Results are shown in Table 4 (results of the above SARS-CoV-2 testing also included). The results demonstrate that the ultra-fast one-pot assay chemistry can broaden the effective temperature range for a variety of different targets.

TABLE 4
Effective Temperature Ranges for Various Targets

| | Conventional Assay 35 | Ultra-Fast Assay, 15 | Ultra-Fast Assay, 15 |
|---|---|---|---|
| Target | min TAT | min TAT (high conc.) | min TAT (near LoD) |
| SARS-CoV-2 | 64-68° C. | 55-70° C. | 60-70° C. |
| Flu A | 60-68° C. | 60-70° C. | 61-70° C. |
| Flu B | 60-67° C. | 55-70° C. | 61-70° C. |
| RSV | 65-68° C. | 65-70° C. | 61-70° C. |
| | (64-68° at higher conc.) | | |
| Pos Ctrl (Beta Actin) | 64-70° C. | 65-70° C. | 62-70° C. |

### Example 6: Real-Time Fluorescence Detection & Quantification

An ultra-fast one-pot RT-LAMP assay was carried out to detect multiple different samples of SARS-CoV-2 RNA using a real-time fluorescent readout. The components used in the assay are shown in Table 5. Total reaction volume was 15 μL.

TABLE 5
Assay with Real-Time Fluorescent Readout

| | Final Reaction | |
|---|---|---|
| Component | Concentration | |
| Primers | (see above) | |
| WarmStart ® Multi-Purpose LAMP/RT-LAMP 2X | 1X | |
| Master Mix with UDG (NEB M1708S)* | | |
| Lyo-Ready Bst DNA Polymerase (Invitrogen ™ | 0.268 | U/μL |
| A56656 or A56657) | | |
| Lyo-Ready SuperScript ™ IV Reverse Transcriptase | 0.333 | U/μL |
| (Invitrogen ™ EP164B1B008 or EP164B2B014) | | |
| GuHCl | 40 | mM |
| RNase inhibitor (RNasin Plus)** | 0.1 | U/μL |
| Fluorescent dye (EvaGreen ® Plus, 20X in water) or | 1X | |
| equivalent | | |
| Trehalose*** | 5% | w/v |

*Includes Bst 2.0 WarmStart ® DNA Polymerase, WarmStart RTx Reverse Transcriptase, dUTP, and thermolabile UDG in a buffer solution. An example alternative is WarmStart ® Multi-Purpose LAMP/RT-LAMP 4X Master Mix with UDG (NEB M1718B).
**Included for assays receiving samples containing nucleases. Can optionally be included up to 2 U/μL or more as needed. Alternative formulation can be RNaseOUT ™ (Invitrogen ™ EO2521B001 or EO2522B001) at 0.1-2 U/μL or more final concentration.
***For lyophilized assays, bring total volume to bead droplet size (16.5 μL in these examples) while achieving desired excipient final concentration. Excipient optionally included up to 10% w/v or more as needed. Alternative excipient formulation can be 10% Trehalose + 4% Dextran (pH 8.1). May also be included in liquid assays (as is the case for the liquid assays of these examples). For liquid assays, nuclease-free water may be added so that the total reaction volume after adding sample (RNA in nuclease-free water or virus in Domus buffer) is 15 μL. For lyobead assays, rehydration volume of 16.5 μL bead is 15 μL.

The reaction mixtures were incubated at a constant temperature of 65° C. for up to 35 minutes, with real-time fluorescence acquisition every 30 seconds monitoring the SYBR Green I channel (SYBR Green I dye=497 nm max excitation, 520 nm max emission; EvaGreen Plus dye=487 nm max excitation, 525 nm max emission).

Positive samples can be distinguished from negatives by monitoring the onset of the exponential phase (i.e., upward inflection point) of their amplification curves. Tailored LAMP kinetic models (e.g., generalized logistic functions or variations) and/or real-time feature extractions (e.g., calculation of first and/or second derivatives) can be applied to analyze the amplification curves to accurately determine the assay's TTP, enabling reliable positive detection, real-time quantification, and differentiation of positives from negatives with short overall turnaround time.

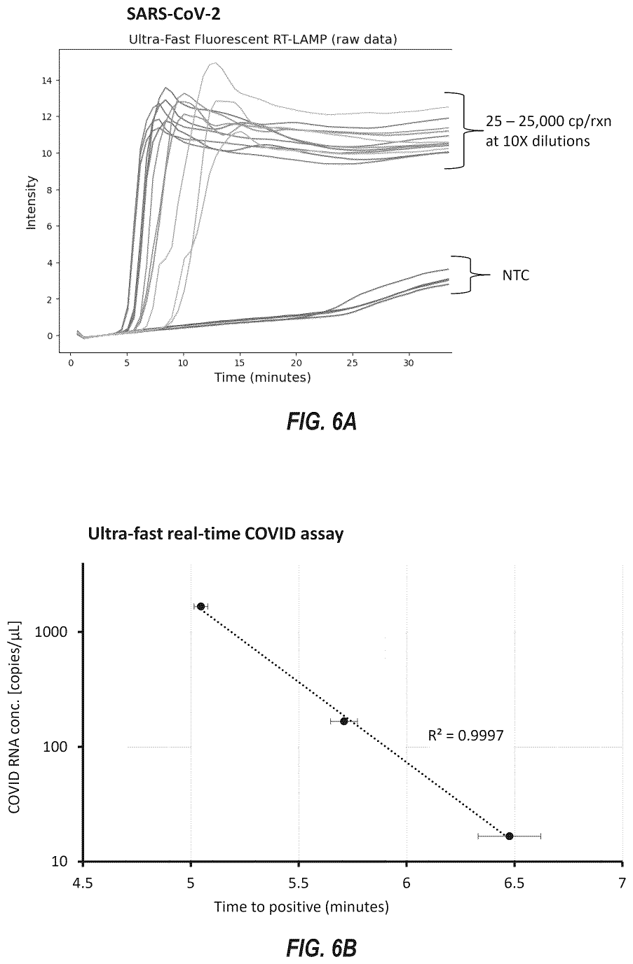

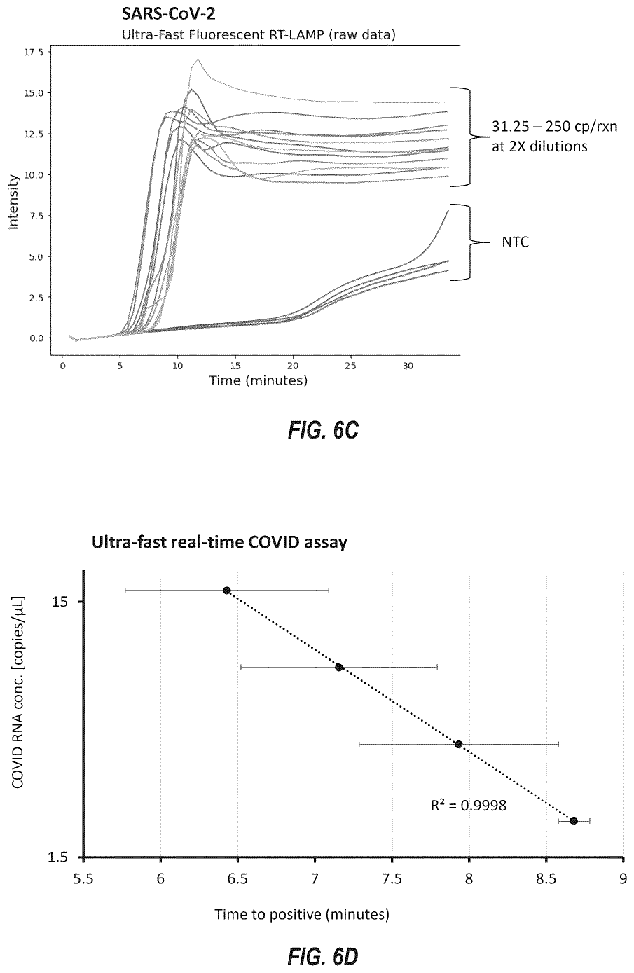

FIGS. 6A-6D show results of testing evaluating the speed, dynamic range, and LoD of the ultra-fast, one-pot fluorescent RT-LAMP assay formulated in this example for targeting SARS-CoV-2. Various concentrations of SARS-CoV-2 RNA were detected with high repeatability, demonstrating the assay's sensitivity, specificity, and real-time quantification capability across a broad linear range.

Target concentration estimates corresponding to a given TTP value can be determined from the log-linear relationship shown in FIGS. 6B and 6D. Some examples based on FIGS. 6B and 6D are shown in Table 6. These are examples only and the TTP-concentration relationship may vary under different experimental conditions (e.g., due to variations in reagents, quantification standard, etc.). With respect to Table 4 and the examples of FIGS. 6B and 6D, other unknown target concentrations, evaluated under the same experimental conditions can readily be determined based on the trendline shown in FIGS. 6B and 6D (which functions similarly to a standard curve in qPCR).

TABLE 6
Expected TTP for Various RNA Concentrations of SARS-CoV-2

| Target Conc. | TTP |
|---|---|
| (cp/μL) | (min) |
| at least 1,667 | 5 or less |
| at least 167 | 5.7 or less |
| at least 16.7 | 6.5 or less |
| at least 8.3 | 7.2 or less |
| at least 4.15 | 7.9 or less |
| at least 2.075 | 8.7 or less |

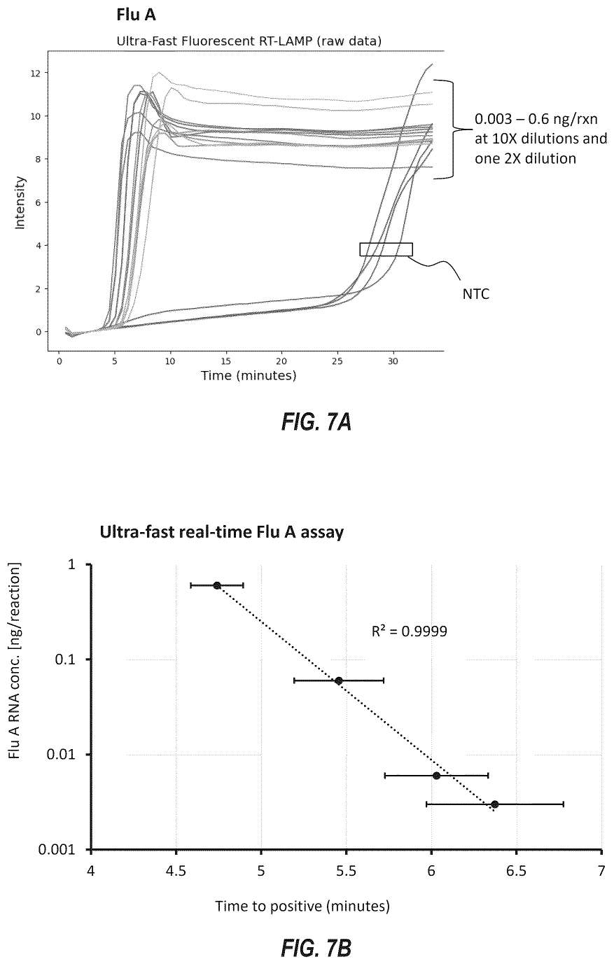

Similar testing was carried out with ultra-fast assays formulated for other targets. FIGS. 7A and 7B show results for the ultra-fast assay targeted to Flu A, with Table 7 showing example expected TTP values based on target concentrations.

TABLE 7
Expected TTP for Various RNA Concentrations of Flu A

| Target Conc. | TTP |
|---|---|
| (ng/rxn) | (min) |
| at least 0.6 | 4.7 or less |
| at least 0.06 | 5.5 or less |
| at least 0.006 | 6.0 or less |
| at least 0.003 | 6.4 or less |

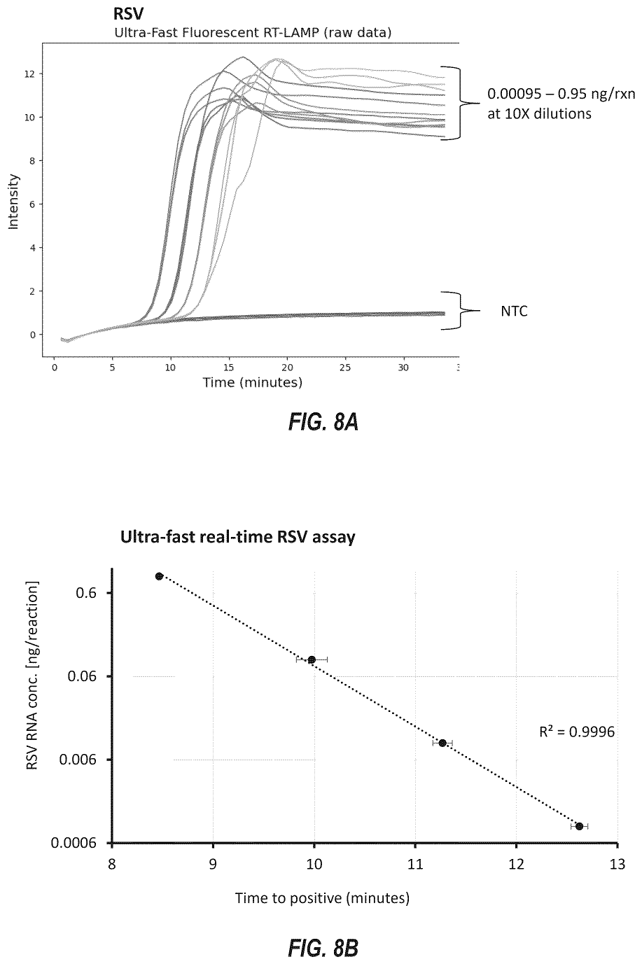

FIGS. 8A and 8B show results for RSV, with Table 8 showing example expected TTP values based on target concentrations.

TABLE 8
Expected TTP for Various RNA Concentrations of RSV

| Target Conc. | TTP |
|---|---|
| (ng/rxn) | (min) |
| at least 0.95 | 8.5 or less |
| at least 0.095 | 10 or less |
| at least 0.0095 | 11.3 or less |
| at least 0.00095 | 12.6 or less |

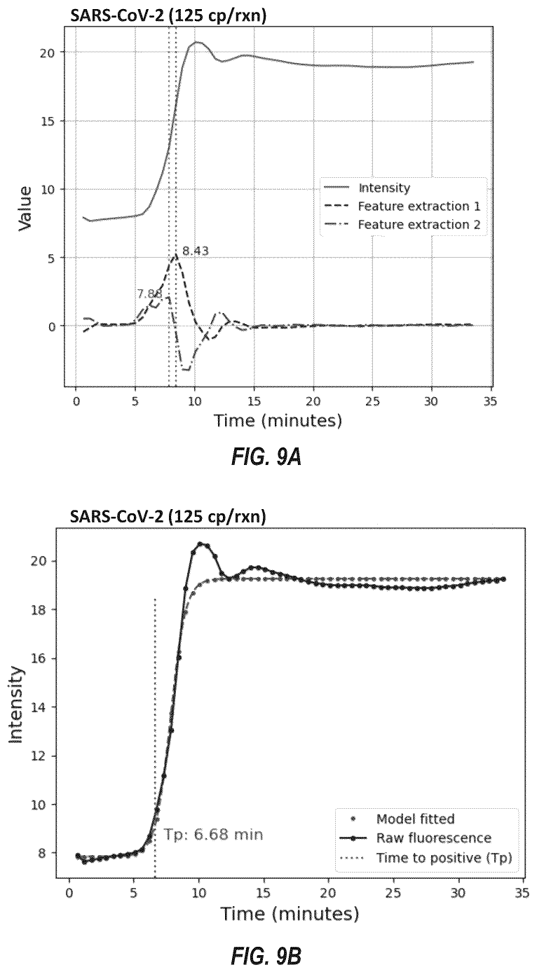

FIGS. 9A and 9B show results of amplification curve analyses of a sample spiked with 125 cp/rxn SARS-CoV-2 RNA. Feature extractions can include, for example, first derivative, second derivative, inflection point, baseline fluorescence, slope of the exponential phase, and/or other feature extraction parameters known in the art. Analyses utilizing mathematical models calibrated with empirical data and tailored to the unique characteristics and kinetics of the ultra-fast one-pot LAMP/RT-LAMP chemistries can also be employed.

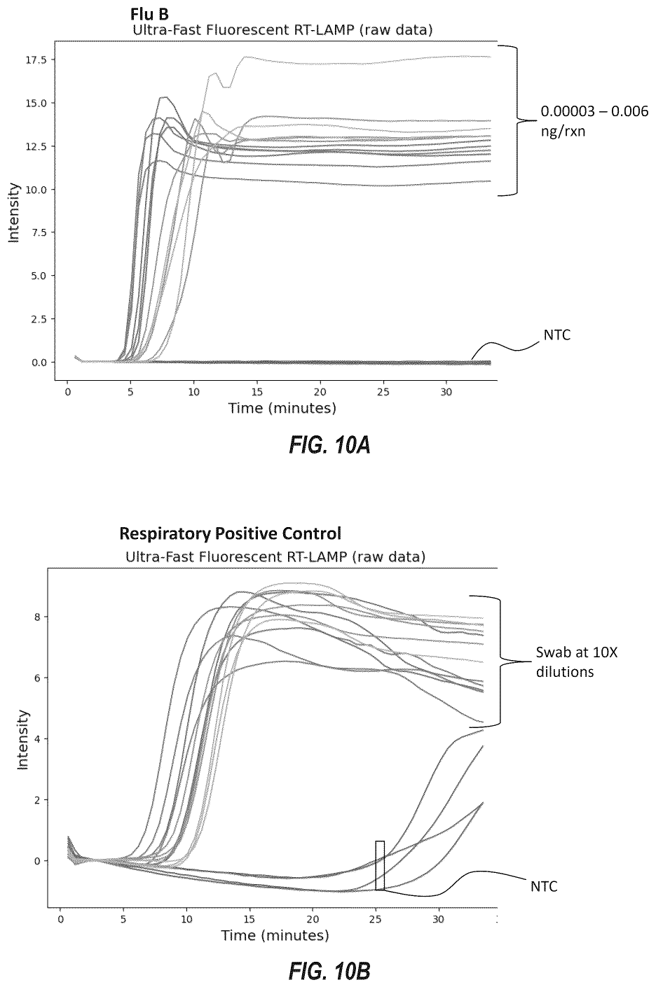

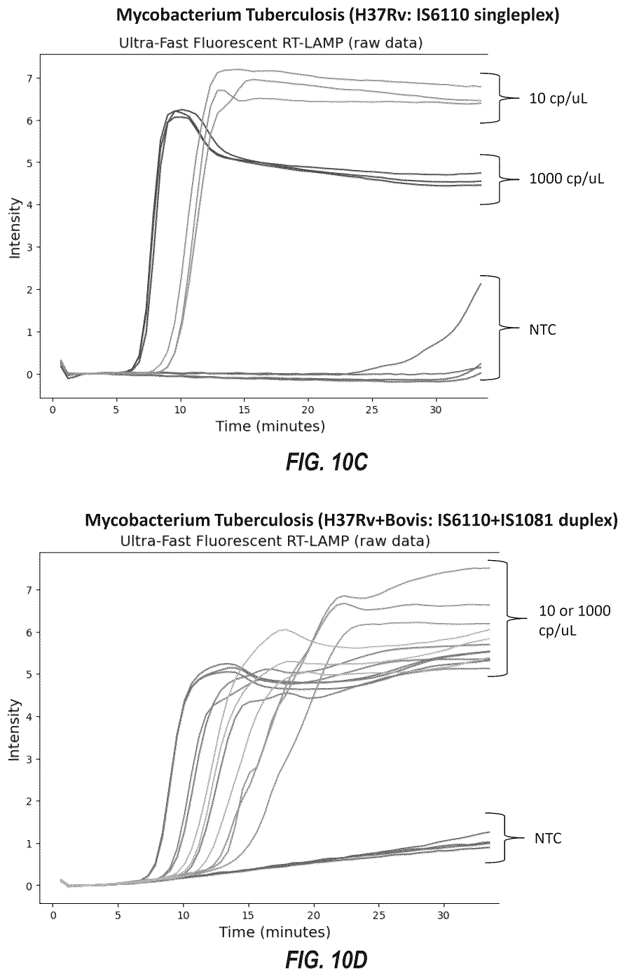

By simply changing the respective primer sets, the ultra-fast, one-pot fluorescent LAMP/RT-LAMP assay was formulated to detect various other targets, as shown by FIGS. 10A-10D.

### Example 7: Alternative Master Mix Formulation

An alternative master mix chemistry, similar to that of Example 1, was formulated as shown in Table 9. Unlike Example 1, the formula of Table 9 does not include a commercial NEB master mix.

| | Final Reaction | |
|---|---|---|
| Component | Concentration | |
| Primers (designed independently for each | (same as Table 1) | |
| respective target) | | |
| Bst Reaction Buffer | 1X | |
| Betaine | 1M | |
| MgCl₂ | 6 | mM |
| RNase inhibitor (RNaseOUT ™) | 1.6 | U/μL |
| dNTP mix | 1.4 mM each | |
| Lyo-Ready Bst DNA Polymerase (Invitrogen ™ | 0.12 | U/μL |
| A56656 or A56657) | | |
| Lyo-Ready SuperScript ™ IV Reverse | 1 | U/μL |
| Transcriptase (Invitrogen ™ EP164B1B008 or | | |
| EP164B2B014) | | |
| GuHCl | 40 | mM |
| 5-Bromo-PAPS | 100 | μM |
| MnCl₂ | 175 | μM |
| Trehalose* | 5% | w/v |

*For lyophilized assays, bring total volume to bead droplet size (16.5 μL in these examples) while achieving desired excipient final concentration. Excipient optionally included up to 10% w/v or more as needed. Alternative excipient formulation can be 10% Trehalose + 4% Dextran (pH 8.1). May also be included in liquid assays (as is the case for the liquid assays of these examples). For liquid assays, nuclease-free water may be added so that the total reaction volume after adding sample (RNA in nuclease-free water or virus in Domus buffer) is 15 μL. For lyobead assays, rehydration volume of 16.5 μL bead is 15 μL.

Testing demonstrated that the Table 9 formulation was as effective as the Table 1 formulation and in some cases, slightly faster.

### Example Aspects

The following list of clauses represent a non-exhaustive list of aspects of the present disclosure:

Clause 1. A composition for performing ultra-fast, one-pot amplification of a target nucleic acid combining ultra-fast loop-mediated isothermal amplification and readout, the composition comprising:
a buffer formulated for receiving a sample, wherein the buffer functions to enable one or more of sample elution, sample dilution, sample storage, sample inactivation, sample lysis, nucleic acid extraction, nucleic acid purification, and nucleic acid stabilization;
an amplification master mix;
a readout indicator mixed with the amplification master mix; and
a set of primers configured for amplification of the target nucleic acid.

Clause 2. The composition of Clause 1, wherein the sample comprises: DNA and/or RNA extracted from microorganisms or other biological samples; processed or unprocessed biological samples; and/or synthetic DNA and/or RNA.

Clause 3. The composition of Clause 2, wherein the sample comprises a lower nasal swab sample, nasopharyngeal swab sample, gingival swab sample, buccal swab sample, gargle sample, sputum sample, and/or saliva sample prepared in a suitable buffer.

Clause 4. The composition of Clause 2, wherein the sample comprises a bodily fluid.

Clause 5. The composition of Clause 2, wherein the sample comprises an environmental sample such as a water and/or soil sample.

Clause 6. The composition of Clause 2, wherein the sample comprises a veterinary sample, agricultural sample, and/or food product sample, such as a feedstock, vegetable, fruit, meat, milk, and/or honey sample.

Clause 7. The composition of Clause 2, wherein the extracted DNA and/or RNA is from a virus, bacteria, algae, and/or other type of microorganism.

Clause 8. The composition of any preceding Clause, wherein the amplification master mix is formulated as a LAMP master mix comprising an Mg salt such as MgSO4 or MgCl2, an NH4 salt such as (NH4)2SO4 or (NH4)2C12, dNTP mix, dUTP, thermolabile UDG and dUTP, strand-displacing DNA polymerase, Tween 20 or Triton-100, betaine, Tris-HCl, and/or KCl.

Clause 9. The composition of Clause 8, wherein the composition enables amplification of the target nucleic acid within a broader effective temperature range as compared to amplification of the target nucleic acid with a composition comprising a conventional master mix.

Clause 10. The composition of Clause 9, wherein the composition enables an effective temperature range of 55° C. to 70° C. for a reaction with a 15-minute turnaround time and/or an effective temperature range of 60° C. to 70° C. for a reaction with a 10-minute turnaround time.

Clause 11. The composition of any one of Clauses 8-10, wherein the strand-displacing DNA polymerase comprises Bst 2.0 and/or Bst 3.0, optionally configured as a warm-start DNA polymerase that is inactive at room temperature.

Clause 12. The composition of any one of Clauses 8-11, wherein the LAMP master mix further comprises a reverse transcriptase, optionally configured as a warm-start reverse transcriptase that is inactive at room temperature.

Clause 13. The composition of Clause 12, wherein the nucleic acid amplification master mix further comprises an additional reverse-transcriptase such as Invitrogen™ Lyo-ready SuperScript™ IV Reverse Transcriptase (P164B1B008 or EP164B2B014) or its equivalent versions (e.g., standard, lyo-ready, glycerol-free, and/or high-concentration).

Clause 14. The composition of any preceding Clause, wherein the nucleic acid amplification master mix further comprises a second strand-displacing DNA polymerase such as a lyophilization-ready Bst DNA Polymerase (Invitrogen™ A56656 or A56657) or its equivalent versions (e.g., standard, lyo-ready, glycerol-free, and/or high-concentration).

Clause 15. The composition of any preceding Clause, wherein the set of primers comprise a forward outer primer (i.e., F3 primer), backward outer primer (i.e., B3 primer), forward inner primer (i.e., FIP primer), and backward inner primer (i.e., BIP primer) formulated to enable amplification of a target nucleic acid sequence from the sample.

Clause 16. The compositions of Clause 15, wherein the set of primers further comprise one or more loop primers (i.e., LoopF primer and/or LoopB primer).

Clause 17. The compositions of Clause 15 or 16, wherein the set of primers further comprise one or more accelerating primers.

Clause 18. The composition of Clause 17, wherein the one or more accelerating primers comprise one or more stem primers, swarm primers, and/or other primers that function by a similar mechanism.

Clause 19. The compositions of any one of Clauses 15-18, wherein the set of primers comprises one or more sets of primers targeting different regions of a specific target nucleic acid sequence and/or targeting different sequences from different target samples.

Clause 20. The composition of any preceding Clause, wherein the readout indicator functions according to: pH, turbidity; fluorescence; nanomaterials; detection of pyrophosphate; detection of metal ions; a lateral flow strip/dipstick mechanism; gel/capillary electrophoresis; microfluidics; microarrays; electrochemical sensors; molecular transducers; or a combination thereof.

Clause 21. The composition of any preceding Clause, wherein the readout indicator comprises a fluorescent indicator such as a double-stranded DNA intercalating dye such as EvaGreen®, EvaGreen® Plus, dsGreen, SYBR® Green I, and/or SYTO 9.

Clause 22. The composition of any preceding Clause, wherein the readout indicator comprises a sequence-specific probe-based fluorescence-generating mechanism, such as a probe with fluorophore-quencher pair, such as molecular beacons, Scorpions probes, strand-displacement probes such as assimilating probes, detection of amplification by releasing of quenching (DARQ) probes, and/or oligonucleotide strand exchange (OSD) probes.

Clause 23. The composition of any preceding Clause, wherein the readout indicator comprises a pH-independent colorimetric indicator comprising a metal indicator that senses metal ions such as Mg2+, Mn2+, Zn2+, Cu2+, Co2+, Cd2+, Fe2+, Ni2+, Hg2+, Pb2+, such as a composition comprising one or more of the metal ions combined with one or more of Hydroxynaphthol Blue, Eriochrome Black T, Calcein, or Pyridylazophenol dye such as 2-(5-Bromo-2-pyridylazo)-5-[N-propyl-N-(3-sulfopropyl)amino]phenol (5-Bromo-PAPS) or 2-(5-Nitro-2-pyridylazo)-5-[N-n-propyl-N-(3-sulfopropyl)amino]phenol (5-Nitro-PAPS).

Clause 24. The composition of any preceding Cla Clause im, wherein the readout indicator comprises a colorimetric double-stranded DNA intercalator such as malachite green and/or methyl green.

Clause 25. The composition of any preceding Clause, wherein the readout indicator comprises a pH-dependent colorimetric indicator including such as Phenol Red, Neutral Red, Cresol Red, Cresol Purple, Thymol Blue, Bromothymol Blue, Bromophenol Blue, Litmus, Chlorophenol Red, Dichlorofluorescein, Methyl Red, Bromocresol Purple, Naphtholphthalein, Cresolphthalein, or combination thereof.

Clause 26. The composition of any preceding Clause, further comprising GuHCl.

Clause 27. The composition of any preceding Clause, wherein the buffer comprises: a surfactant such as Tween 20, Tween 80, Triton X-100, Triton X-114, NP-40, Igepal CA-630, CHAPS, and/or SDS; a reducing/denaturing agent such as DTT, TCEP, urea, GITC, and/or formamide; a nucleases inhibitor such as proteinase K, murine RNase inhibitor, VSA, PVSA, ACP, RNasin Plus Ribonuclease Inhibitor, RiboGrip RNase Inhibitor, RiboLock RNase Inhibitor, SUPERase•In, RNaseOUT™, and/or RNAsecure; a chelating agent such as EDTA; a molecular enhancer such as polyethylene glycol; and/or a buffering salt such as Tris-HCl, TE, TAE, TBE, and/or a solution of HCl, NaOH or KOH.

Clause 28. The composition of Clause 27, wherein the buffer comprises Tween 20 at 0.05% to 2.5% w/v, such as 0.1% w/v, and urea at 5 mM to 25 mM, such as 10 mM, prepared in nuclease-free water.

Clause 29. The composition of any preceding Clause, wherein the master mix comprises one or more excipients such as sucrose, trehalose, dextran, pullulan, lactose, glucose, raffinose, mannitol, sorbitol, glycine, histidine, arginine, gelatin, dextrose, hydroxyethyl starch, poly(ethylene glycol), poly(propylene glycol), poly(vinyl alcohol), polyvinylpyrrolidone (PVP), or combination thereof, such as comprising trehalose at 5% to 20% w/v, such as 10% w/v, and dextran at 2% to 20% w/v, such as 3% to 10% w/v, such as 4% w/v, optionally controlled to a pH of 8.1.

Clause 30. The composition of any preceding Clause, wherein the amplification master mix is formulated in one-pot liquid solution form.

Clause 31. The composition of any one of Clauses 1-29, wherein the amplification master mix is one-pot freeze-dried (i.e., lyophilized) or air-dried.

Clause 32. The composition of Clause 30 or 31, wherein the one-pot amplification master mix includes the set of primers.

Clause 33. The composition of any preceding Clause, wherein the readout indicator is formulated to provide quantitative, semi-quantitively, or qualitative readout for the reaction.

Clause 34. A method for performing ultra-fast, one-pot amplification of a target nucleic acid, the method combining ultra-fast loop-mediated isothermal amplification and readout, the method comprising: mixing a sample with a one-pot reaction mixture comprising a nucleic acid amplification master mix, a set of nucleic acid primers, and at least one readout indicator; subjecting the reaction mixture within a reaction vessel to thermal incubation at a substantially constant temperature for a period of time, during which (i) the sample is processed, (ii) target nucleic acids are amplified, and (iii) a readout indicator is activated.

Clause 35. The method of Clause 34, wherein the sample is prepared in a buffer that functions to enable one or more of sample elution, sample dilution, sample storage, sample inactivation, sample lysis, nucleic acid extraction, nucleic acid purification, or nucleic acid stabilization.

Clause 36. The method of Clause 34 or 35, wherein the method utilizes a composition as in any one of Clauses 1-33.

Clause 37. The method of any one of Clauses 34-36, wherein the sample or portion thereof is added to the one-pot reaction mixture by pipetting, microfluidics, microcapillaries, wicking by a porous media or material, or combinations thereof, either manually, semi-automatically, or automatically.

Clause 38. The method of any one of Clauses 34-37, wherein the one-pot reaction mixture is lyophilized or air-dried and is reconstituted by the sample or portion thereof to a desired total reaction volume.

Clause 39. The method of any one of Clauses 34-38, wherein the readout indicator comprises a fluorescent indicator and wherein the method further comprises quantifying the target nucleic based on resulting fluorescence, such as via mathematical modeling and/or feature extraction of a resulting amplification curve.

Clause 40. A kit for performing ultra-fast, one-pot amplification of a target nucleic acid combining ultra-fast loop-mediated isothermal amplification and readout, the kit comprising: a sample collection device; a first vessel that contains a sample processing buffer; a second vessel that contains an aqueous, frozen, air-dried, or lyophilized nucleic acid amplification master mix, the master mix comprising a set of nucleic acid primers and a readout indicator for nucleic acid amplification and result readout; a device for transferring a portion of a sample from the first vessel into the second vessel; and optionally, a heat source that generates heat with a temperature profile suitable for causing both the sample processing and the nucleic acid amplification reaction.

Clause 41. The kit of Clause 40, wherein the kit utilizes a composition as in any one of Clauses 1-33 and/or is utilized in a method as in any one of Clauses 34-39.

Clause 42. The kit of Clause 40 or 41, wherein the heat source is battery-powered, USB-powered, solar-powered, chemically powered by an exothermic reaction, or combination thereof.

Clause 43. The kit of any one of Clauses 40-42, wherein the heat source comprises both a mechanism for generating the heat and a mechanism for regulating the output temperature profile.

Clause 44. The kit of any one of Clauses 40-43, further comprising a container configured with sufficient insulation and proper venting to maintain a desired output temperature profile over a period of time needed for carrying out the reaction.

1. A composition for performing ultra-fast, one-pot amplification of a target nucleic acid combining ultra-fast loop-mediated isothermal amplification and readout, the composition comprising:
a buffer formulated for receiving a sample, wherein the buffer functions to enable one or more of sample elution, sample dilution, sample storage, sample inactivation, sample lysis, nucleic acid extraction, nucleic acid purification, and nucleic acid stabilization;
an amplification master mix;
a readout indicator mixed with the amplification master mix; and
a set of primers configured for isothermal amplification of the target nucleic acid.

2. The composition of claim 1, wherein the sample comprises:
DNA and/or RNA extracted from microorganisms or other biological samples;
processed or unprocessed biological samples; and/or
synthetic DNA and/or RNA.

3. The composition of claim 2, wherein the sample comprises a lower nasal swab sample, nasopharyngeal swab sample, gingival swab sample, buccal swab sample, gargle sample, sputum sample, and/or saliva sample prepared in a suitable buffer.

4. The composition of claim 1, wherein the amplification master mix comprises: an Mg salt; an NH₄ salt; dNTP mix; dUTP; thermolabile UDG and dUTP; strand-displacing DNA polymerase; Tween 20 or Triton-100; betaine; Tris-HCl; and/or KCl.

5. The composition of claim 4, wherein the composition enables amplification of the target nucleic acid within a broader effective temperature range as compared to amplification of the target nucleic acid with a composition comprising a conventional master mix.

6. The composition of claim 4, wherein the strand-displacing DNA polymerase comprises Bst 2.0 and/or Bst 3.0, optionally configured as a warm-start DNA polymerase that is inactive at room temperature.

7. The composition of claim 4, wherein the amplification master mix further comprises a reverse transcriptase, optionally configured as a warm-start reverse transcriptase that is inactive at room temperature.

8. The composition of claim 7, wherein the nucleic acid amplification master mix further comprises an additional reverse-transcriptase.

9. The composition of claim 4, wherein the amplification master mix further comprises a second strand-displacing DNA polymerase.

10. The composition of claim 4, further comprising GuHCl.

11. The compositions of claim 11, wherein the set of primers comprises one or more accelerating primers.

12. The composition of claim 1, wherein the readout indicator comprises a fluorescent double-stranded DNA intercalating dye.

13. The composition of claim 1, wherein the readout indicator comprises a pH-independent colorimetric indicator comprising a metal indicator that senses metal ions.

14. The composition of claim 1, wherein the readout indicator comprises a pH-dependent colorimetric indicator.

15. The composition of claim 1, wherein the buffer comprises:
a surfactant comprising Tween 20, Tween 80, Triton X-100, Triton X-114, NP-40, Igepal CA-630, CHAPS, and/or SDS;
a reducing/denaturing agent comprising DTT, TCEP, urea, GITC, and/or formamide;
optionally, a nucleases inhibitor comprising proteinase K, murine RNase inhibitor, VSA, PVSA, ACP, RNasin Plus Ribonuclease Inhibitor, RiboGrip RNase Inhibitor, RiboLock RNase Inhibitor, SUPERase•In, RNaseOUT™, and/or RNAsecure;
optionally, a chelating agent comprising EDTA;
optionally, a molecular enhancer comprising as polyethylene glycol; and/or
optionally, a buffering salt comprising Tris-HCl, TE, TAE, TBE, and/or a solution of HCl, NaOH or KOH.

16. The composition of claim 15, wherein the buffer comprises Tween 20 at 0.05% to 2.5% w/v and urea at 5 mM to 25 mM, prepared in nuclease-free water.

17. The composition of claim 1, wherein the master mix comprises trehalose at 5% to 20% w/v and dextran at 2% to 20% w/v, optionally controlled to a pH of 8.1.

18. The composition of claim 1, wherein the amplification master mix is formulated in one-pot liquid solution form.

19. The composition of claim 1, wherein the amplification master mix is one-pot freeze-dried (i.e., lyophilized) or is air-dried.

20. A method for performing ultra-fast, one-pot amplification of a target nucleic acid, the method combining ultra-fast loop-mediated isothermal amplification and readout, the method comprising:
mixing a sample with a one-pot reaction mixture comprising a nucleic acid amplification master mix, a set of nucleic acid primers, and at least one readout indicator;
subjecting the reaction mixture within a reaction vessel to thermal incubation at a substantially constant temperature for a period of time, during which (i) the sample is processed, (ii) target nucleic acids are amplified, and (iii) a readout indicator is activated.
